(19) United States
(12) Patent Application Publication
(10) Pub. No.: US 2005/0165452 A1
(43) Pub. Date: Jul. 28, 2005

(54) Antithrombogenic medical device

(75) Inventors: Daniel C. Sigg, St. Paul, MN (US); James A. Coles JR., Minneapolis, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc.

(21) Appl. No.: 10/766,792

(22) Filed: Jan. 28, 2004

(51) Int. Cl. A61N 1/18; A61N 1/30; A61N 1/372; A61N 1/39

(52) U.S. Cl. 607/6

(57) ABSTRACT

A therapy delivery and/or diagnostic device includes a layer of a catalytic agent, having nitrite reductase and/or nitrate reductase, or nitrosothiol reductase activity. The catalytic layer converts nitrate/nitrate or nitrosothiols to nitric oxide when in contact with blood.

### TECHNICAL FIELD

The present invention relates to implantable therapy delivery and/or diagnostic devices and more particularly to such devices that include a blood-contacting surface adapted to generate nitric oxide (NO).

### BACKGROUND

Cardiac rhythm management (CRM) systems often employ a therapy delivery and/or diagnostic device coupled to a surface of a patient's heart via one or more medical electrical leads. Typically the one or more leads include electrodes for both stimulating the heart and sensing electrical activity of the heart. Alternatively, or in addition to the electrodes, leads may include means for sensing physiological parameters, such as pressure or blood oxygen content, and/or means for therapeutic and/or diagnostic fluid infusion.

After a period of time, implanted devices become encapsulated by fibrotic tissue, the process begun in part by thrombus generation at blood-contacting surfaces of the implanted device. Implantable materials including means to release NO in order to simulate antithrombogenic properties of endothelial cells have been generally proposed for incorporation in a host of medical devices. It is desirable to incorporate such a material into a device in order to keep active surfaces of the device free from encapsulation, which may inhibit function of the device, and/or to increase ease of chronic explant of the devices.

Although embodiments of the present invention are described in the context of cardiac implants, it should be recognized that the scope of the invention includes any implantable medical device including blood-contacting surfaces.

### BRIEF DESCRIPTION OF THE DRAWINGS

The following drawings are illustrative of particular embodiments of the invention and therefore do not limit its scope, but are presented to assist in providing a proper understanding of the invention. The drawings are not to scale (unless so stated) and are intended for use in conjunction with the explanations in the following detailed description. The present invention will hereinafter be described in conjunction with the appended drawings, wherein like numerals denote like elements, and:

FIG. 1 is a schematic rendering of an implanted exemplary CRM system, which may incorporate one or more embodiments of the present invention;

FIG. 2 is a plan view of one of the devices included in the CRM system shown in FIG. 1;

FIGS. 3A-C are plan views with partial sections of portions of devices according to some embodiments of the present invention;

FIGS. 4A-C are plan views with partial sections of portions of devices according to some alternate embodiments of the present invention;

FIG. 5 is a radial section view of a device according to another embodiment of the present invention;

FIG. 6 is a plan view of another exemplary device which may incorporate one or more embodiments of the present invention;

FIG. 7A is a plan view with a partial section of a portion of a device according to another embodiment of the present invention;

FIG. 7B is a radial section view through section line A-A of the device shown in FIG. 7A;

FIG. 8A is a plan view with a partial section of a portion of a device according to another embodiment of the present invention; and

FIG. 8B is a plan view with a partial section of a portion of a device according to yet another embodiment of the present invention.

### DETAILED DESCRIPTION

The following detailed description is exemplary in nature and is not intended to limit the scope, applicability, or configuration of the invention in any way. Rather, the following description provides a practical illustration for implementing exemplary embodiments of the invention.

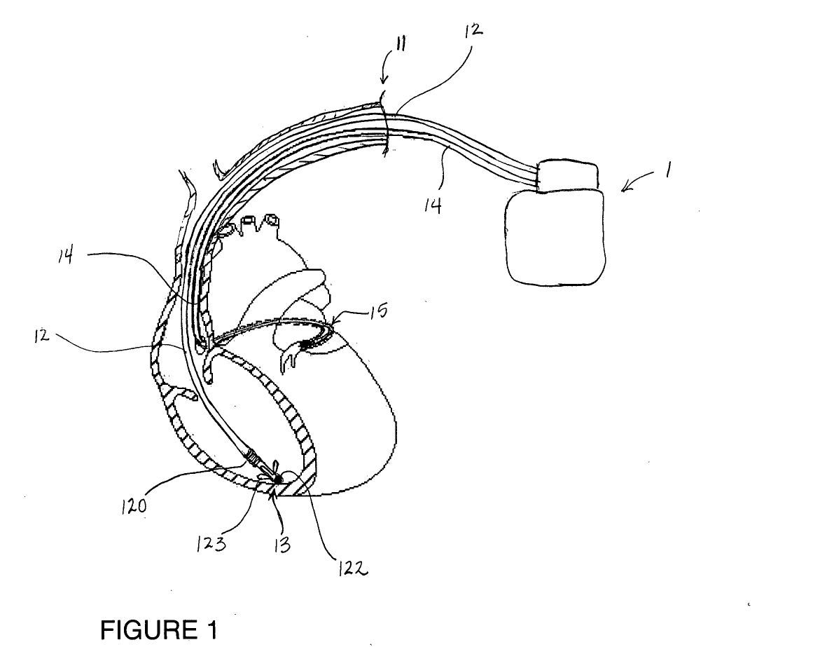

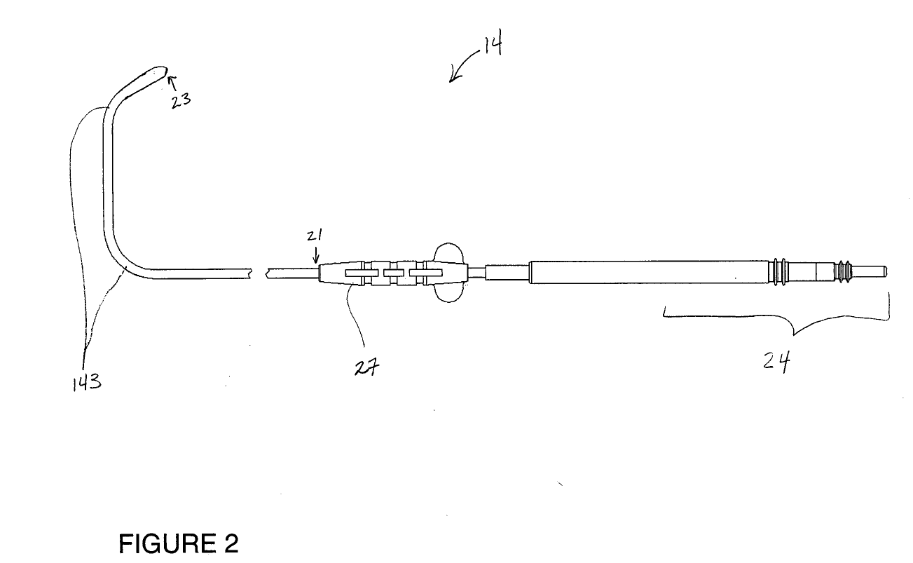

FIG. 1 is a schematic rendering of an implanted exemplary CRM system, which may incorporate one or more embodiments of the present invention. FIG. 1 illustrates the CRM system including a generator 1 to which a first device 12 and a second device 14 are coupled and extend therefrom into a patients vascular system to implant sites 15 and 13, respectively, entering via a vascular entry site 11. Means for coupling devices 12 and 14 to generator 1 are well known to those skilled in the art, one example of which is via an IS-1 connector 24, illustrated in FIG. 2, inserted into a connector module port of generator 1.

FIG. 1 further illustrates first device 12 including a stimulating tip electrode 122 and a coil electrode 120, which may either be an anode acting in conjunction with tip electrode 122 (cathode), a high voltage defibrillation electrode, or a combination of both; a fixation element 123, in the form of tines, holds tip electrode 122 in contact with tissue at implant site 13. FIG. 2 is a plan view of device 14 illustrating an alternate fixation element composed of two preformed bends 143 which serve to hold device 14 at implant site 15 within a cardiac vein; either an electrode (not shown) or an infusion port (not shown) is positioned in proximity to a distal end 23 of device 14 in order to deliver therapy to implant site 15.

According to embodiments of the present invention a layer of catalytic agent capable of converting nitrite/nitrate or nitrosothiols to nitric oxide, when in contact with blood, is present on an outer surface of a polymeric layer overlaying a portion of device 12 in proximity to implant site 13 and/or device 14 in proximity to implant site 15, wherein the portion in proximity to implant site 13, 15 is defined in conjunction with FIG. 1 as any portion between vascular entry site 11 and implant site 13, 15. (FIG. 2 further illustrates device 14 including an anchoring sleeve 27 defining a point 21 along device 14 which may approximately correspond with vascular entry site 11 when device 14 is implanted.) The catalytic agent according to one embodiment is a biocatalytic agent and according to an alternate embodiment is a biomimetic agent; both agents are described by Batchelor et al. in U.S. Patent application 2002/0115559, which is incorporated by reference in its entirety herein. Batchelor et al. further describe means for attaching the catalytic agents to substrates, including adsorption, covalent bonding and the like. According to one embodiment, a portion of device 12 or 14, in the form of a polyurethane outer sheath, is covered with a Cu(II)-complex doped film, which is formed in part from 132 mg polyurethane and 4 mg of Cu(II) metal ion ligand complex, a biomimetic agent.

In addition to an outer layer of catalytic agent, some embodiments of the present invention further include a polymeric substrate/matrix underlying the layer of catalytic agent, which contains a reservoir of lipophilic salts or nitrite/nitrate or nitrosothiols that can leak to the catalytic layer; such is also described by Batchelor et al. in the aforementioned patent application. In the ensuing description, layers of catalytic agent and reservoirs in polymeric substrates, which are incorporated into various embodiments of the present invention, include any of those described in Batchelor et al.

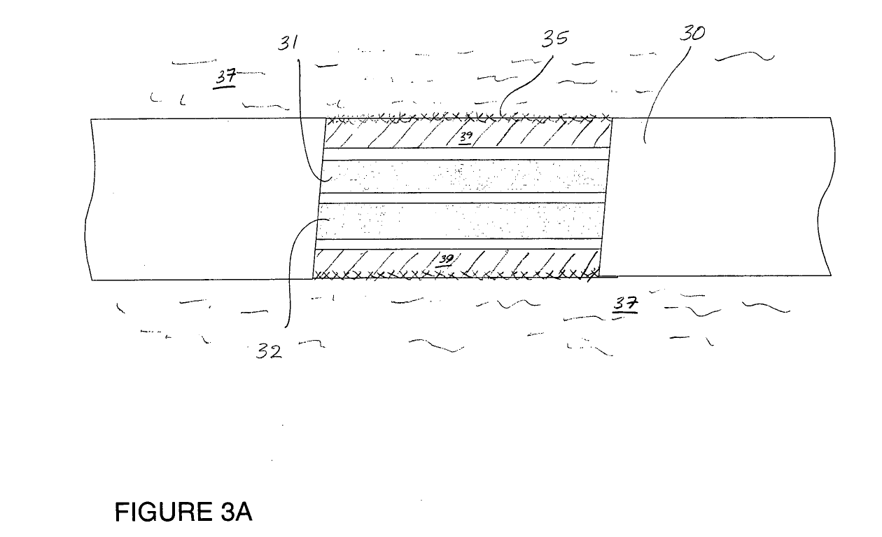

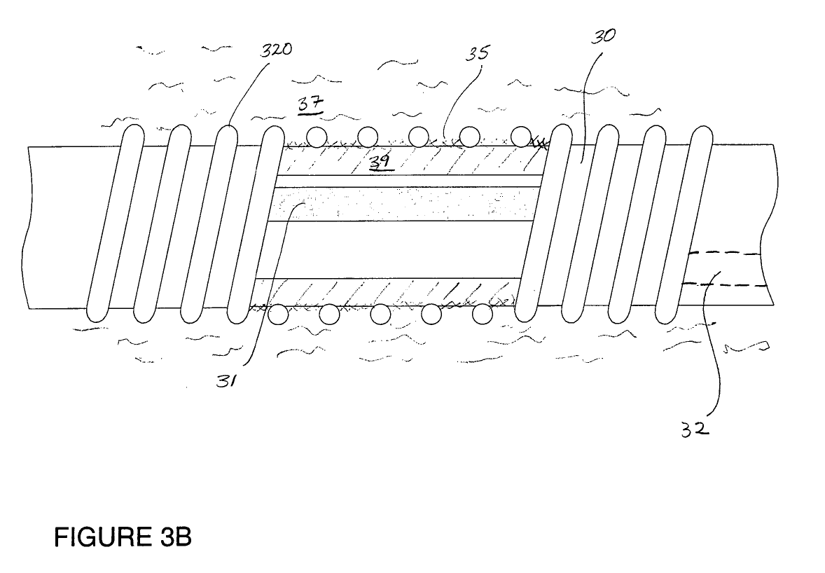

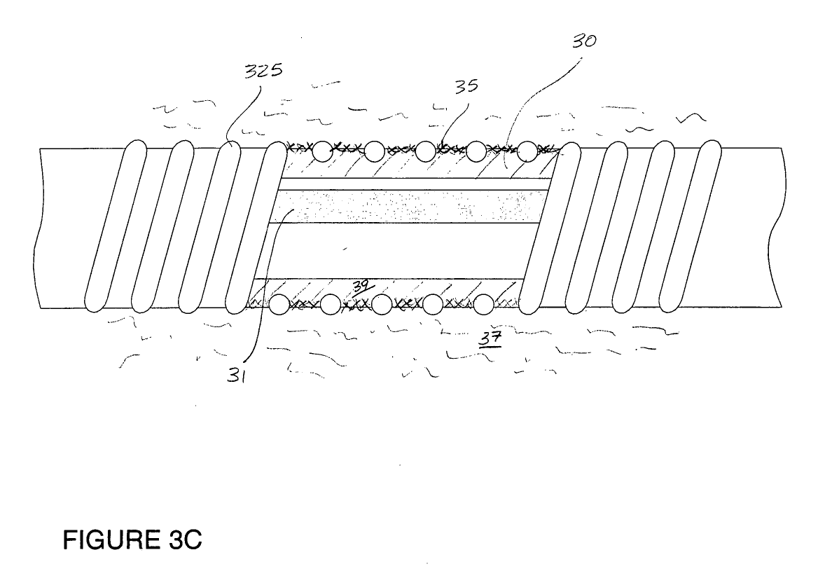

FIGS. 3A-C are plan views with partial sections of portions of devices according to embodiments of the present invention. FIG. 3A illustrates a polymer layer 30, for example an outer sheath, surrounding a first conductor 31 and a second conductor 32 extending therethrough; polymer layer 30 includes a layer of catalytic agent 35, present on an outer surface thereof and forming an interface with a surrounding blood pool/stream 37. Catalytic agent 35 may be present along an entire length of device 12 and or 14 (FIG. 1), a discrete segment of the portion of device 12 and or 14 that extends within the vascular system, or a length of device 12 and or 14 extending from venous entry 11 approximately to implant site 15 or 13, respectively. According to one embodiment of the present invention, polymer layer 30 forms a device body to carry conductors 31, 32; examples of materials forming layer 30 include silicone and polyurethane.

FIGS. 3B-C illustrate layer of catalytic agent 35 extending beneath coil electrode 320, 325, which is mounted on the device body and coupled to conductor 32; either an end of coil 320, 325 extends inward through layer 30 to conductor 32 or conductor 32 extends outward to coil 320, 325 to be coupled, for example, by crimping, welding or other methods known to those skilled in the art. According to the embodiment illustrated in FIG. 3B, coil electrode 320 overlays the outer surface of polymer layer 30, while, according to the embodiment illustrated in FIG. 3C, electrode 325 is embedded in polymer layer 30. In either case, catalytic agent 35 can contact surrounding blood 37 in between turns of coil 320, 325 to convert nitrite/nitrate or nitrosothiols to nitric oxide. According to some embodiments, layer of catalytic agent 35 is only present on polymer layer 30 in that area corresponding to electrode 320, 325, as illustrated, (or coil 120 illustrated in FIG. 1) since this area is particularly susceptible to thrombus formation due to surface discontinuities caused by coil 320, 325. Alternately, as previously described, catalytic agent 35 is further present along portions of polymer layer 30 extending away from coil 320, 325, 120.

FIGS. 3A-C further illustrate polymer layer 30 including a bulk matrix or substrate 39 underlying layer of catalytic agent 35. According to some embodiments of the present invention, as previously described, bulk matrix 39 includes a reservoir of lipophilic salts or nitrite/nitrate or nitrosothiols that can leak to catalytic layer 35. Alternate embodiments are contemplated wherein the catalytic agent is dispersed throughout layer 30, for example having been blended into bulk matrix 39 of layer 30 during an initial forming process or having been absorbed into bulk matrix 39 in a secondary process.

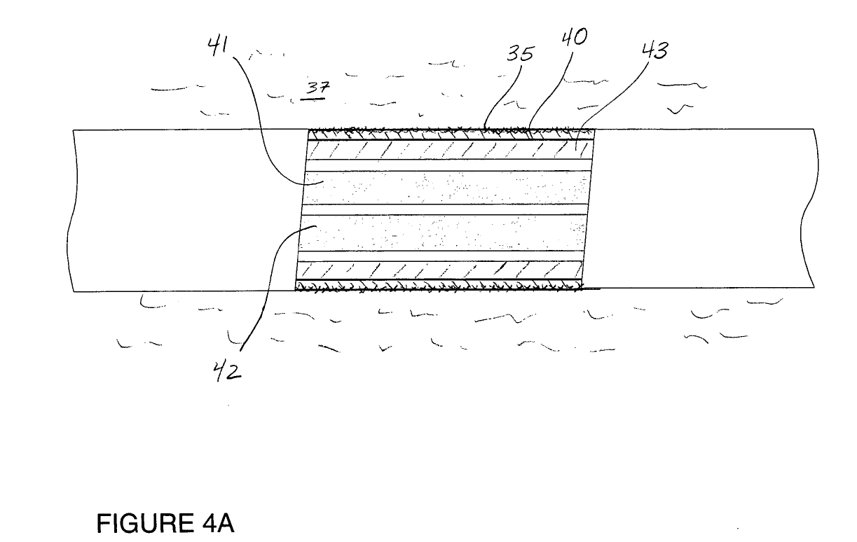

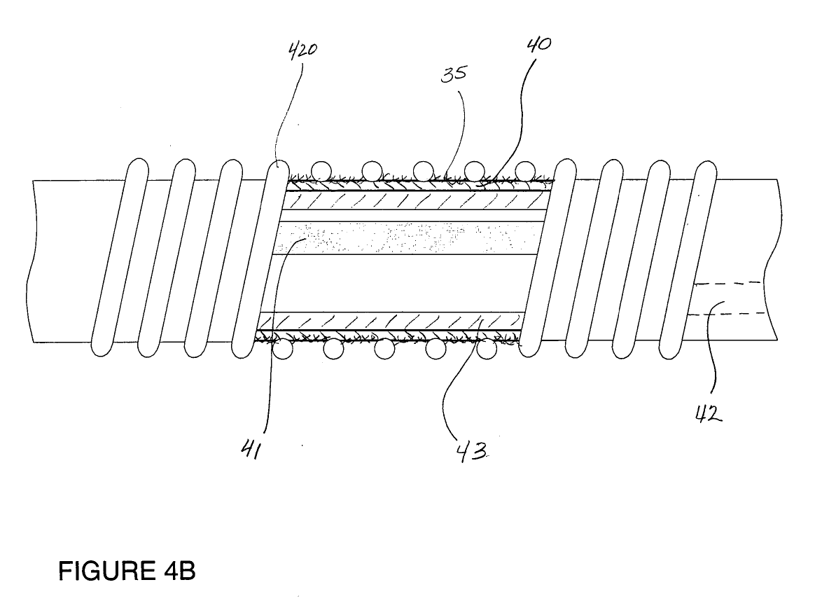

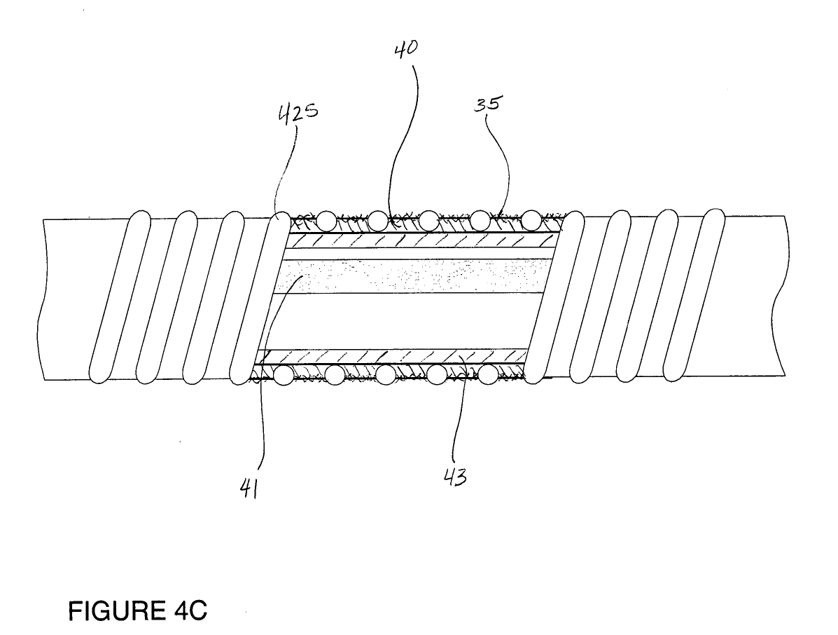

FIGS. 4A-C are plan views with partial sections of portions of devices according to alternate embodiments of the present invention. FIG. 4A illustrates a polymer layer 40 including layer of catalytic agent 35, present on an outer surface thereof and forming an interface with surrounding blood 37. In contrast to the embodiment illustrated in FIG. 3A, polymer layer 40, rather than forming a device body, as layer 30 does, is a separate element overlaying a device body 43. Examples of materials forming layer 40 include silicone, polyurethane and PTFE. Layer 40 including catalytic agent 35 may extend along all or a portion of device 12 and or 14 (FIG. 1). According to one embodiment, layer 40 extends up to coil electrode 120 and the outer surface of layer 40 is approximately isodiametric with an OD of coil 120; such a construction is described in commonly assigned U.S. Pat. No. 6,052,625, for example in conjunction with FIGS. 3 and 9 of that patent, the descriptions of which are incorporated by reference herein. According to alternate embodiments, layer 40 including catalytic agent 35 underlies coil electrodes as is illustrated in FIGS. 4B-C.

FIG. 4B illustrates a coil 420 mounted on device body 43 and overlaying the outer surface of layer 40, while FIG. 4C illustrates a coil 425 mounted on device body 43 and embedded in layer 40; coil 420, 425 is coupled to conductor 42 in a manner previously described in conjunction with FIGS. 3B-C. In either case, catalytic agent 35 can contact surrounding blood 37 in between turns of coil 420, 425 to convert nitrite/nitrate or nitrosothiols to nitric oxide. According to some embodiments, layer of catalytic agent 35 is only present on polymer layer 40 in that area corresponding to electrode 420, 425, as illustrated, since this area is particularly susceptible to thrombus formation as was previously described in conjunction with FIGS. 3B-C. Alternately, as previously described, catalytic agent 35 is further present along portions of polymer layer 40 extending away from coil 420, 425.

Although not labeled in FIGS. 4A-C, polymer layer 40, similar to layer 30, includes a bulk matrix or substrate underlying layer of catalytic agent 35. As previously described, according to some embodiments of the present invention the bulk matrix includes a reservoir of lipophilic salts or nitrite/nitrate or nitrosothiols that can leak to catalytic layer 35; and, according to alternate embodiments, the catalytic agent is dispersed throughout layer 40.

It should be noted that catalytic layer 35 illustrated in FIGS. 3B-C and FIGS. 4B-C may be formed on outer surfaces of layers 30 and 40, respectively either before or after coil electrodes 320, 325 and 420, 425, respectively, are mounted on the device bodies. Further, according to some embodiments, layer of catalytic agent 35 comprising a biomimetic agent, which is a metal ion ligand complex, is covalently attached to outer surfaces of coil electrodes 320, 325 and 420, 425.

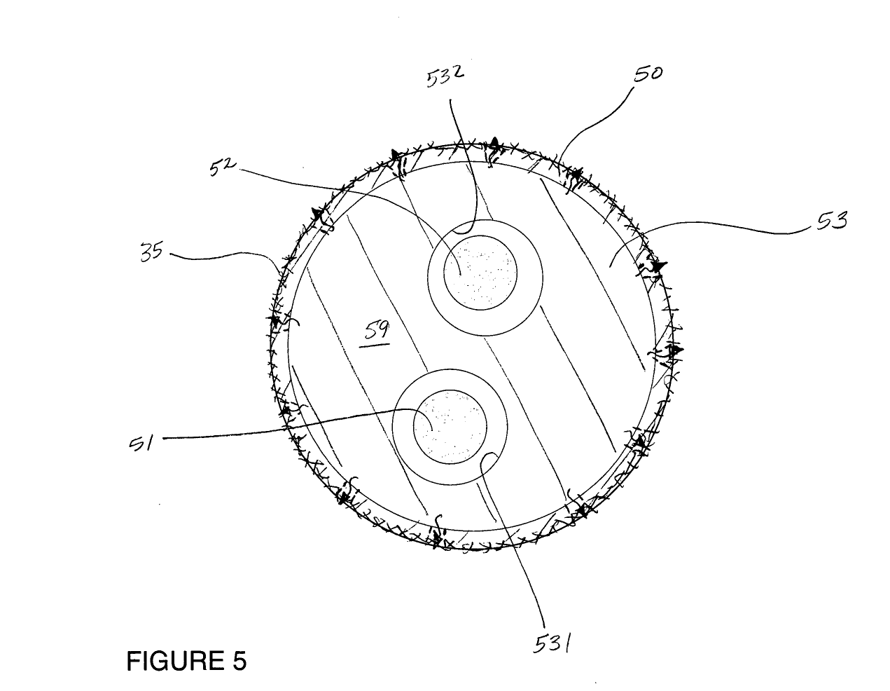

Conductors 31, 41 and 32, 42, according to some embodiments, include one or more electrically conductive wires, examples of which include, but are not limited to a cable formed of a plurality of MP35N wires and a coil formed of one or more MP35N wires. Conductors 31, 41 and 32, 42 of this type are electrically isolated from one another via insulative layers formed about each conductor 31, 41 and 32, 42 or according the embodiment illustrated in FIG. 5.

FIG. 5 is a radial section view of a device according to another embodiment of the present invention. FIG. 5 illustrates a device body 53 in the form of a multilumen tube including a first lumen 531 carrying a first conductor 51 and a second lumen 532 isolated from first lumen 531 and carrying a second conductor 52. FIG. 5 further illustrates a polymer layer 50 overlaying device body 53 and including layer of catalytic agent 35 on outer surface of layer 50, similar to embodiments described in conjunction with FIGS. 4A-C. According to other embodiments, layer 50 is not included and device body 53 is the polymer layer on which layer of catalytic agent 35 present, similar to embodiments described in conjunction with FIGS. 3A-C.

FIG. 5 further illustrates yet another embodiment wherein dashed lines represent a plurality of pores through which lipophilic salts or nitrite/nitrate or nitrosothiols leak to catalytic layer 35 from a reservoir held in a bulk matrix 59 of device body 53. According to an alternate embodiment catalytic layer 35 is present on outer surface of device body 53, rather than on outer surface of layer 50, and is in communication with outer surface of layer 50 via the plurality of pores. Such embodiments of layer 50 including the plurality of pores may be formed, for example, from expanded-PTFE. Furthermore, according to yet another embodiment, layer 50 including the plurality of pores extends over a coil electrode, for example electrodes 320, 325, the pores in this case allowing electrical conduction therethrough. It should be noted that, in yet another embodiment, layer 50, including the plurality of pores and overlaying an electrode, may be replaced by a conductive polymer layer, which performs the similar function of allowing electrical conduction and includes a layer of catalytic agent 35 on an outer surface thereof.

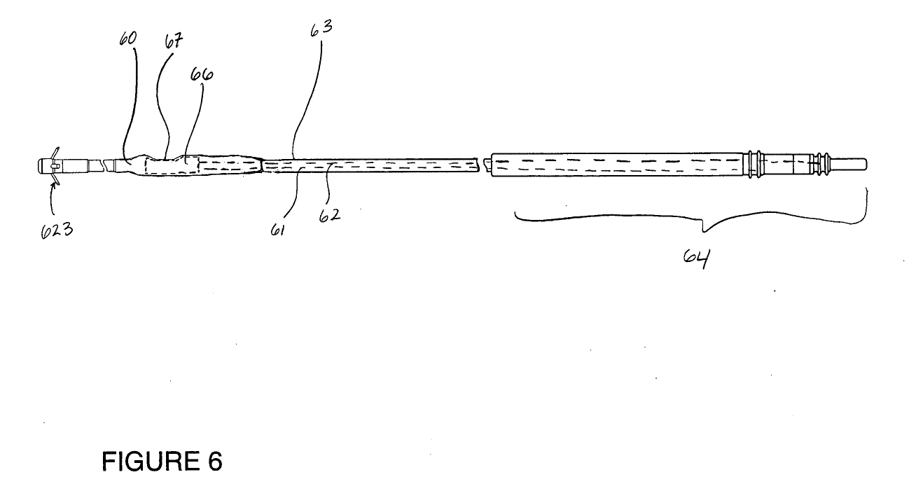

FIG. 6 is a plan view of another exemplary device, which may incorporate one or more embodiments of the present invention. FIG. 6 illustrates a device body 63 extending from a connector 64 and carrying a first conductor 61 and a second conductor 62, which electrically couple a sensor capsule 66 to contacts on connector 64; a tine fixation element 623, positioned in proximity to sensor capsule 66, is adapted to secure the device to an implant site. FIG. 6 further illustrates a polymer layer 60 overlaying a portion of sensor capsule 66; sensor capsule includes an active surface 67, which according to one embodiment is a diaphragm adapted to transmit blood pressure forces, one example of which is described in commonly assigned U.S. Pat. No. 5,564,434 which is incorporated by reference herein. According to some embodiments of the present invention polymer layer 60 includes a layer of catalytic agent adapted to convert nitrite/nitrate or nitrosothiols to NO, as previously described, which is attached to an outer surface of layer 60 surrounding sensor capsule active surface 67. Thus, active surface 67, due to NO production in surrounding blood, may be kept free of thrombotic attachments, which could hinder performance of surface 67.

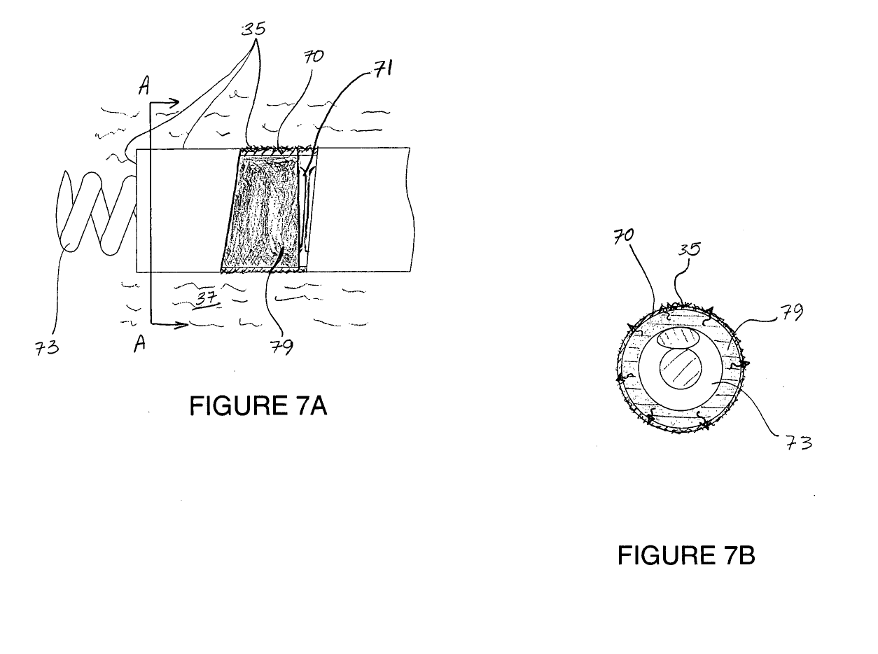

FIG. 7A is a plan view with a partial section of a distal portion of a device according to another embodiment of the present invention and FIG. 7B is a radial section view through section line A-A of FIG. 7A. FIG. 7A illustrates a helical fixation element 73 extending from the distal portion and coupled to a conductor 71 extending within a polymer layer 70, which includes layer of catalytic agent 35 attached to an outer surface thereof; element 73 is adapted to secure the device to an implant site and further serves as an electrode to stimulate tissue in proximity to the implant site. Element 73 along with other electrodes described herein may be formed from any appropriate material known to those skilled in the art, one example of which is platinum. According to embodiments of the present invention, catalytic agent 35 is adapted to convert nitrite/nitrate or nitrosothiols, in blood near implant site, to NO, as previously described. Furthermore, according to another aspect of the present invention, NO formed in proximity to an electrode-tissue interface may increase an electrical efficiency of the interface by inhibiting proliferative and/or inflammatory responses of tissue cells.

FIG. 7A further illustrates a polymeric plug 79, which may be formed from polyurethane or silicone, held within polymer layer 70, wherein, according to some embodiments, polymer plug 79 contains within its bulk matrix a reservoir of lipophilic salts or nitrite/nitrate or nitrosothiols that can leak to catalytic layer 35 in close proximity to implant site thereby increasing NO generation, which may further enhance an electrical stimulating interface between tissue and element 73 which is embedded therein at implant. Furthermore, element 73 may include a steroid coating, for example beclomethasone diproprionate, which also serves to enhance the electrical interface according to means well known to those skilled in the art of cardiac pacing. According to other embodiments, plug 79 includes catalytic layer 35, which is exposed to blood 37 via a plurality of pores included in layer 70, similar to that described in conjunction with FIG. 5.

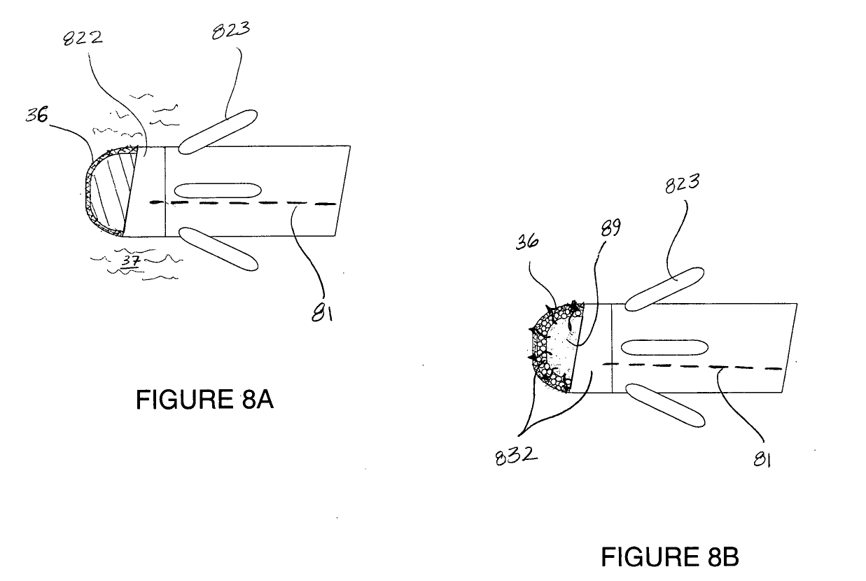

FIG. 8A is a plan view with a partial section of a distal portion of a device according to another embodiment of the present invention. FIG. 8A illustrates a tine fixation element 823 in proximity to a tip electrode 822, which is coupled to a conductor 81 and includes a layer of catalytic agent 36 comprising a biomimetic agent, which is a metal ion ligand complex, attached to an outer surface thereof, for example by covalent bonding. According to embodiments of the present invention, catalytic agent 36 is adapted to convert nitrite/nitrate or nitrosothiols, in blood 37 near implant site, to NO, in order to enhance an electrical interface between electrode 822 and tissue at the implant site, as previously described. FIG. 8B is a plan view with a partial section of a portion of a device according to yet another embodiment of the present invention. FIG. 8B illustrates a porous electrode 832 formed, for example, by a sintering process known to those skilled in the art and including layer of catalytic agent 36; electrode 832 contains a polymer plug 89, which may be formed from polyurethane or silicone. According to the illustrated embodiment, polymer plug 89 holds within its bulk matrix a reservoir of lipophilic salts or nitrite/nitrate or nitrosothiols that can leak to catalytic layer 36, through pores of electrode 832, in close proximity to implant site thereby increasing NO generation, which may further enhance an electrical stimulating interface as was previously described in conjunction with FIGS. 7A-B. According to an alternate embodiment, plug 89 holds a steroid which may elute over time through porous electrode 832; such a construction for steroid elution through a porous electrode is well known to those skilled in the art and may be modified to incorporate the alternate embodiment of plug 89, which holds a reservoir of lipophilic salts or nitrite/nitrate or nitrosothiols. According to further embodiments a porous layer, such as that forming electrode 832, overlays layer of catalytic agent 36, which may be either incorporated into plug 89 or formed on electrode 822 illustrated in FIG. 8A, such that agent 36 can contact surrounding blood 37 through the pores.

In the foregoing detailed description, the invention has been described with reference to specific embodiments. However, it may be appreciated that various modifications and changes can be made without departing from the scope of the invention as set forth in the appended claims.

1. An implantable therapy delivery and/or diagnostic device, comprising:
a fixation element adapted to secure the device to an implant site;
one or more elongate conductors extending within the device;
a polymeric layer overlaying a portion of the device in proximity to the implant site and including an outer surface; and
a layer of a catalytic agent, having nitrite reductase and/or nitrate reductase, or nitrosothiol reductase activity, present on the outer surface of the polymeric layer;
wherein the catalytic layer converts nitrite/nitrate or nitrosothiols to nitric oxide when in contact with blood.

2. The device of claim 1 wherein the polymeric layer is formed of a material selected from the group consisting of silicone, polyurethane, PTFE and expanded PTFE.

3. The device of claim 1, wherein the polymeric layer further includes a bulk matrix containing a reservoir of lipophilic salts or nitrite/nitrate or nitrosothiols that can leak to the layer of catalytic agent.

4. The device of claim 1, further comprising an elongate body, which carries the one or more conductors, and wherein the polymeric layer forms the device body.

5. The device of claim 4, wherein the polymeric layer is a multilumen tube.

6. The device of claim 4, further comprising a coil electrode coupled to a one of the one or more conductors and overlaying the outer surface of the polymeric layer; wherein the one of the one or more conductors includes an electrically conductive wire.

7. The device of claim 4, further comprising a coil electrode coupled to a one of the one or more conductors and partially imbedded in the outer surface of the polymeric layer; wherein the one of the one or more conductors includes an electrically conductive wire.

8. The device of claim 1, further comprising an elongate body, which carries the one or more conductors, and wherein the polymeric layer overlays the device body.

9. The device of claim 8, wherein the device body is a multilumen tube.

10. The device of claim 8, further comprising a coil electrode coupled to a one of the one or more conductors and overlaying the outer surface of the polymeric layer; wherein the one of the one or more conductors includes an electrically conductive wire.

11. The device of claim 8, further comprising a coil electrode coupled to a one of the one or more conductors and partially embedded in the outer surface of the polymeric layer; wherein the one of the one or more conductors includes an electrically conductive wire.

12. The device of claim 8, wherein the polymeric layer includes a plurality of pores extending therethrough and the device body contains a reservoir of lipophilic salts or nitrite/nitrate or nitrosothiols which can leak to the layer of catalytic agent.

13. The device of claim 8, further comprising a coil electrode coupled to a one of the one or more conductors and overlaying the device body; wherein the one of the one or more conductors includes an electrically conductive wire and wherein the polymeric layer extends over the coil electrode and allows electrical conduction therethrough.

14. The device of claim 8, wherein the polymeric layer further includes a bulk matrix containing a reservoir of lipophilic salts or nitrite/nitrate or nitrosothiols that can leak to the layer of catalytic agent.

15. The device of claim 1, further comprising:
a physiological sensor capsule coupled to the one or more conductors;
wherein the outer surface of the polymeric layer overlays a portion of the sensor capsule; and
the one or more conductors includes an electrically conductive wire.

16. The device of claim 1, further comprising a polymeric plug held within the polymeric layer, the polymeric plug containing a reservoir of lipophilic salts or nitrite/nitrate or nitrosothiols that can leak to the layer of catalytic agent.

17. The device of claim 1, further comprising:
a distal tip electrode coupled to a one of the one or more conductors and adapted to stimulate the implant site;
a polymeric plug held within the polymeric layer and containing a reservoir of lipophilic salts or nitrite/nitrate or nitrosothiols that can leak to the layer of catalytic agent;
wherein the layer of catalytic agent is positioned in close proximity to the tip electrode; and
the one of the one or more conductors includes an electrically conductive wire.

18. The device of claim 17, wherein the polymeric plug is formed of a material selected from the group consisting of silicone and polyurethane.

19. The device of claim 1, wherein the catalytic agent comprises a biocatalytic agent.

20. The device of claim 1, wherein the catalytic agent comprises a biomimetic catalytic agent.

21. The device of claim 20, wherein the biomimetic catalytic agent comprises a Cu(II) metal ion ligand complex.

22. An implantable medical electrical lead comprising:
a distal fixation element adapted to secure the medical electrical lead to an implant site;
one or more elongate electrical conductors;
an electrode coupled to a one of the one or more conductors, adapted to stimulate in proximity to the implant site and including an outer surface; and
a layer of a catalytic agent, having nitrite reductase and/or nitrate reductase, or nitrosothiol reductase activity, attached to the outer surface of the electrode;
wherein the catalytic layer converts nitrite/nitrate or nitrosothiols to nitric oxide when in contact with blood

23. The lead of claim 22, wherein the electrode further includes a porous side wall and further comprising a polymeric plug held within the electrode side wall; the plug containing a reservoir of lipophilic salts or nitrite/nitrate or nitrosothiols that can leak through the porous sidewall to the layer of catalytic agent.

24. The lead of claim 23, wherein the polymeric plug is formed of a material selected from the group consisting of silicone and polyurethane.

25. The lead of claim 22, wherein the catalytic agent comprises a metal ion ligand complex.

26. The lead of claim 22, further comprising a porous layer overlaying the layer of catalytic agent.

27. An implantable therapy delivery and/or diagnostic device comprising:
a body including a sidewall having a plurality of pores;
a plug held within the porous sidewall and including a layer of catalytic agent, having nitrite reductase and/or nitrate reductase, or nitrosothiol reductase activity present on an outer surface of the plug;
wherein the catalytic layer, exposed to blood through the plurality of pores, converts nitrite/nitrate or nitrosothiols to nitric oxide when in contact with the blood.
